(19) United States
(12) Patent Application Publication
(10) Pub. No.: US 2006/0161122 A1
(43) Pub. Date: Jul. 20, 2006

(54) Absorbent article with multi-zone acquisition

(75) Inventors: Edward Erdman, West Chester, PA (US); Dane Jackson, Gilbertsville, PA (US); Elizabeth Miller, King of Prussia, PA (US)

Correspondence Address: GOSZ AND PARTNERS, LLP, 450 BEDFORD STREET, LEXINGTON, MA, 02420, US

(73) Assignee: TYCO HEALTHCARE RETAIL GROUP, AG

(21) Appl. No.: 11/299,290

(22) Filed: Dec. 8, 2005

Related U.S. Application Data
(60) Provisional application No. 60/634,963, filed on Dec. 10, 2004.

(51) Int. Cl. A61F 13/15

(52) U.S. Cl. 604/378; 604/361

(57) ABSTRACT

An absorbent article is provided that includes a liquid pervious top layer, a liquid impervious back layer and an absorbent core disposed therebetween. A plurality of acquisition members are disposed between the top layer and the core. The acquisition members are disposed parallel with a longitudinal length of the core in a configuration that retards strikethrough time. A portion of the top layer may be deformed to form a predetermined pattern. The predetermined pattern may be in spatial alignment with the acquisition members in a configuration that retards strikethrough time. Alternatively, the absorbent article includes a film layer disposed between the core and the back layer.

### RELATED APPLICATIONS

This application claims the benefit of U.S. Provisional Application No. 60/634,963, filed Dec. 8, 2004.

### BACKGROUND

1. Technical Field

The present disclosure generally relates to absorbent articles, and more particularly, to a disposable absorbent article, such as, for example, training pants, which include structure that promotes toilet training and contains fluid discharge and body waste.

2. Background of the Related Art

Absorbent articles such as, for example, training pants, disposable diapers, adult incontinent pads, sanitary napkins, pantiliners, incontinent garments, etc. are generally worn, in cooperation with garments and disposed against a body surface by infants or adult incontinent individuals. The absorbent article is employed to collect and absorb body fluid discharge, such as, for example, blood, menses, urine, aqueous body fluids, mucus and cellular debris. For example, the absorbent article may be disposed between the legs of an individual adjacent a crotch area. The absorbent article is positioned with a garment and drawn into engagement with a body surface of the crotch area to collect fluid discharge.

As is known, absorbent articles typically include a fluid permeable coverstock for engaging the body surface, a fluid impermeable backsheet and an absorbent core supported therebetween. The backsheet serves as a moisture barrier to prevent fluid leakage to the garment. The absorbent core usually includes a liquid retention material that faces the body surface. The absorbent core can include, for example, loosely formed cellulosic fibers, such as, for example, wood pulp, fluff pulp, etc. for acquiring and storing fluid discharge. Elasticized regions can be provided around the edges of the article to secure the article about the waist and legs of a wearer.

During toilet training for children, training pants are employed as a training aid. A known training technique is to employ a training pant or underwear garment so that a child feels discomfort following fluid discharge and body waste. Such a technique is employed to provide motivation for proper toilet training.

Some of these known types of training pants, however, suffer from poor fluid discharge and body waste containment. This can disadvantageously result in wetting and staining of the child's environment, e.g., furniture and clothing.

It would therefore be desirable to overcome the disadvantages and drawbacks of the prior art by providing an absorbent article, including a plurality of acquisition members, that promotes toilet training, and contains fluid discharge and body waste.

### SUMMARY

Accordingly, an absorbent article, including a plurality of acquisition members, that promotes toilet training, and contains fluid discharge and body waste is disclosed. Objects and advantages of the present disclosure are set forth in part herein and in part will be obvious therefrom, or may be learned by practice of the present disclosure that is realized and attained by the instrumentalities and combinations pointed out in the appended claims for the devices and methods of the present disclosure comprising its constituent parts, constructions, arrangements, combinations, steps and improvements herein shown and described.

In one particular embodiment, an absorbent article is provided that includes a liquid pervious top layer and a liquid impervious back layer. An absorbent core is disposed between the top layer and the back layer. A plurality of acquisition members are disposed between the top layer and the core. The acquisition members are disposed parallel with a longitudinal length of the core in a configuration that retards strikethrough time. The absorbent article may further include one or a plurality of acquisition members such as, for example, four acquisition members.

The acquisition members may extend an entire length of the core. Alternatively, the acquisition members can extend only a portion of the length of the core. The acquisition members may include non-woven material. The acquisition members may provide visual indicia. The acquisition members can be in contacting engagement. Alternatively, the acquisition members are in spaced apart relation. The acquisition members may be spaced apart a distance in a range of 1 mm to 30 mm. Each of the acquisition members can have a width in a range of 10 mm to 70 mm wide.

A portion of the top layer may be mechanically deformed to form a predetermined pattern. The predetermined pattern may be in spatial alignment with the acquisition members. Alternatively, the absorbent article includes a film layer disposed between the core and the back layer.

In an alternate embodiment, a plurality of acquisition members are disposed between the top layer and the core. The acquisition members are disposed parallel with a longitudinal length of the core and in spatial alignment with the predetermined pattern in a configuration that retards strikethrough time.

In another alternate embodiment, a disposable training absorbent article is provided that includes a liquid pervious top layer having leg gathers disposed adjacent lateral edges thereof. A portion of the top layer is mechanically deformed to form a predetermined pattern of puckers. An absorbent core is disposed between the top layer and a back layer. A plurality of acquisition members are spaced apart and disposed between the top layer and the core. The acquisition members are disposed parallel with a longitudinal length of the core and in spatial alignment with the puckers in a configuration that retards strikethrough time.

### BRIEF DESCRIPTION OF THE DRAWINGS

The objects and features of the present disclosure are set forth with particularity in the appended claims. The present disclosure, as to its organization and manner of operation, together with further objectives and advantages may be understood by reference to the following description, taken in connection with the accompanying drawings, in which:

FIG. 1 is a plan view of an absorbent article in accordance with the principles of the present disclosure;

FIG. 2 is a perspective view, with parts separated, of the absorbent article shown in FIG. 1;

FIG. 3 is a side schematic cross section view of selected portions of the absorbent article shown in FIG. 1;

FIG. 4 is a side schematic cross section view of an alternate embodiment of the absorbent article shown in FIG. 3;

FIG. 5 is a plan view of an alternate embodiment of the absorbent article shown in FIG. 1; and

FIG. 6 is a plan view of another alternate embodiment of the absorbent article shown in FIG. 1.

### DETAILED DESCRIPTION OF EXEMPLARY EMBODIMENTS

The exemplary embodiments of the absorbent article and methods of use disclosed are discussed in terms of absorbent articles, and more particularly, in terms of a disposable absorbent article, such as, for example, training pants, including a plurality of acquisition members, that promotes toilet training, and contains fluid discharge and body waste. The presently described absorbent article is configured to retard strikethrough time for fluid discharge in accordance with the principles of the present disclosure. It is contemplated that the training pants described may be employed as a training aid with for example, a young child, accident victim, elderly person, etc. to promote toilet training.

In the discussion that follows, the term “body-facing surface” refers to a portion of a structure that is oriented towards a body surface, and the “garment-facing surface” refers to a portion of the structure that is oriented towards a garment and is typically opposing the body-facing surface and may be referred to as such. As used herein, the term “body surface” refers to a portion of an individual's body that the absorbent article is disposed with for collecting and absorbing fluid discharge from the individual. As used herein, the term “absorbent article,” “absorbent garment” or “garment” refers to garments that absorb and contain body waste, and more specifically, refers to garments that are placed against or in proximity to the body of the wearer to absorb and contain the various body discharges and wastes.

A non-exhaustive list of examples of absorbent articles includes training pants, diapers, diaper covers, disposable diapers, feminine hygiene products and adult incontinence products. The term absorbent articles includes all variations of absorbent garments, including disposable absorbent garments that are intended to be discarded or partially discarded after a single use and unitary disposable absorbent garments that have essentially a single structure. As used herein, the term “training pants” refers to an absorbent article generally worn by children about the lower torso. The absorbent article may be disposable or partially discarded after a single use. The absorbent article can be a unitary structure or include replaceable inserts or interchangeable parts.

Absorbent articles and diapers may have a number of different constructions. In each of these constructions it is generally the case that an absorbent core is disposed between a liquid pervious, body-facing topsheet and a liquid impervious, exterior facing backsheet. In some cases, one or both of the topsheet and backsheet may be shaped to form a pant-like article. In other cases, the topsheet, backsheet and absorbent core may be formed as a discrete assembly that is placed on a main chassis layer and the chassis layer is shaped to form a pant-like absorbent article. The absorbent article may be provided to the consumer in the fully assembled pant-like shape or may be partially pant-like and require the consumer to take the final steps necessary to form the final pant-like shape. Training pants are typically fully formed and applied by pulling the absorbent article over a child's legs. For a diaper application, an open article is provided and wrapped about a child's waist, such as by manually fastening one or more fasteners or fastener tabs.

The following discussion includes a description of the absorbent article in accordance with the present disclosure. Reference will now be made in detail to the exemplary embodiments of the disclosure, which are illustrated in the accompanying FIGURES.

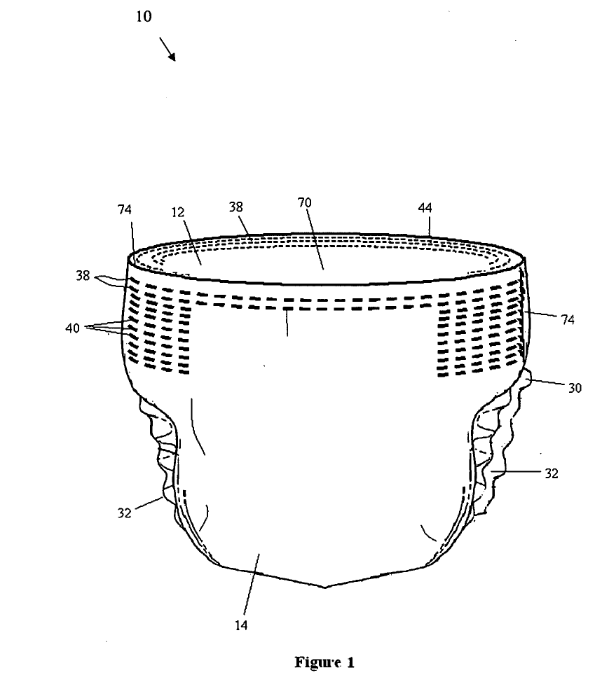

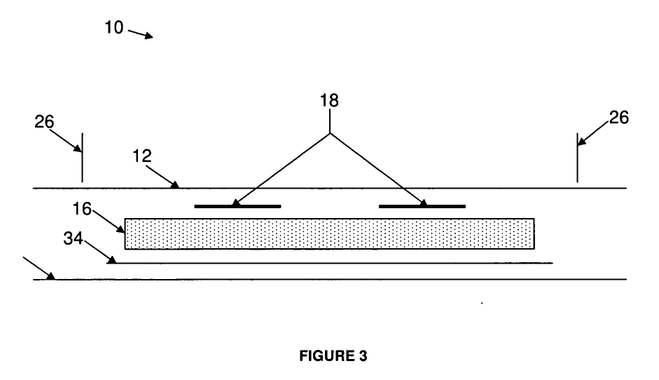

Turning now to the FIGURES, wherein like components are designated by like reference numerals throughout the several views. Referring to FIGS. 1-3, there is illustrated an absorbent article, constructed in accordance with the principles of the present disclosure, such as, for example training pants 10. Training pants 10 includes a liquid pervious top layer 12 and a liquid impervious back/chassis layer 14. An absorbent core 16 is disposed between top layer 12 and back/chassis layer 14. Training pants 10 define a longitudinal axis x and a lateral axis y. It is envisioned that one or a plurality of tissue layers may be disposed about core 16.

A plurality of acquisition members 18 are disposed between top layer 12 and core 16. Acquisition members 18 are disposed parallel with a longitudinal length, disposed along longitudinal axis x, of core 16 in a configuration that retards strikethrough time. Acquisition members 18 are configured to form multi zone acquisition layers that run in parallel relation and are split or spaced apart from each other. It is envisioned that acquisition members 18 may be spaced apart a distance in the range of 1 millimeter (mm) to 30 mm. The configuration of acquisition members 18 slow strikethrough times and allow the wearer to feel wetter for a shorter period of time, while allowing dry out to maintain health of the skin of the wearer. This configuration advantageously promotes toilet training, and contains fluid discharge and body waste.

Acquisition members 18 may be fabricated from various suitable materials, such as nonwoven BICO air bond, adhesive bond, thermal bond, apertured film or airlaid materials, or any combination thereof. Acquisition members 18 can provide visual indicia such as being colored, have a printed pattern and embossed, or may include a three dimensional apertured film with colored pigment such as blue for boys and pink for girls. It is envisioned that acquisition members 18 may be disposed in contacting engagement. Acquisition members 18 may be variously sized and configured, such as, for example, circular, rectangular, elliptical, etc. It is envisioned that acquisition members 18 may define multi acquisition zones and each member having a width in the range of 10 mm to 70 mm. Although two acquisition members 18 are shown, it is contemplated that one or a plurality of acquisition members 18 may be employed with training pants 10. Alternatively, training pants 10 may include four acquisition members 18. It is further contemplated that acquisition members 18 may be disposed in various spatial orientations such as, for example, non-parallel, offset, converging, diverging, etc.

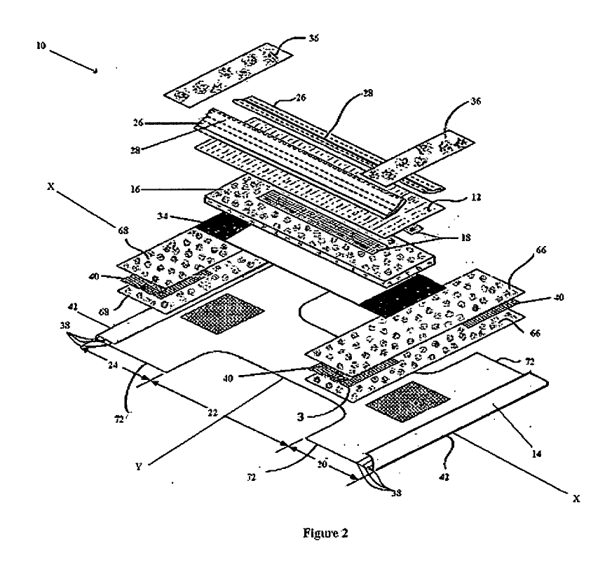

Training pants 10 has a chassis with a generally hourglass shape, as shown by back/chassis layer 14 in FIG. 2. The chassis is defined by a front waist region 20, a crotch region 22 and a back waist region 24. This configuration is shaped to form a pants-like garment when worn by the wearer, as shown in FIG. 1. Those skilled in the art will recognize that “front” and “back” are relative terms, and these regions may be transposed without departing from the scope of the present disclosure. Alternatively, the chassis can be configured in a generally rectangular shape or in a “T” shape.

Top layer 12 and back/chassis layer 14 are different sizes, however, alternatively, they may be substantially coterminous. The terms “top layer” and “back layer” denote the relationship of these materials or layers with respect to absorbent core 16. It is contemplated that additional layers may be present between top layer 12 and back/chassis layer 14, examples of which being described herein. Top layer 12 and back/chassis layer 14 are bonded about their perimeter regions, encasing absorbent core 16 in place. It is envisioned that top layer 12 and back/chassis layer 14 may be indirectly joined by intermediary parts to maintain the structural integrity of training pants 10. It is further envisioned that the various parts of training pants 10 are connected to maintain structural integrity by various known methods such as, for example, adhesives such as hot melt adhesives and construction adhesives, chemical or solvent bonding, ultrasonic welding, stitching, heat bonding, or other suitable known methods of affixation.

Top layer 12 and back/chassis layer 14 may be fabricated from various known materials suitable for applications in accordance with the principles of the present disclosure. For example, top layer 12 may include synthetic fibers (e.g., polypropylene or polyester fibers), natural fibers (e.g., wood or cellulose), apertured plastic films, reticulated foams, porous foams and non-woven liner sheet materials that readily allow passage of liquids to core 16. Suitable liner sheet materials include non-woven spun bond or carded webs of polypropylene, polyethylene, nylon, polyester and blends of these materials. Top layer 12 may have various properties such as being fluid pervious and hydrophobic, which may be imparted by treatment with adhesives, surfactants, or other chemicals. Top layer 12 may also be made from a laminate of overlaid sheets of material, and treated with skin wellness ingredients such as aloe, vitamin E, and the like.

Back/chassis layer 14 may include films of polyethylene, polypropylene, polyester, nylon, polyvinyl chloride, blends of these materials and be pigmented for color. Back/chassis layer 14 may further include separate regions having different properties, such as, air-permeable portions to improve breathability and comfort. Back/chassis layer 14 may be formed from microporous poly coverstock for added breathability. Alternatively, back/chassis layer 14 may form a composite of different sheet materials, chemically treated, heat treated, or by other processes known in the art. It is contemplated that back/chassis layer 14 may include fluid pervious regions. It is further contemplated that back/chassis layer 14 may also be made from a laminate of overlaid sheets of material.

Back/chassis layer 14 may be covered with an additional fibrous, non-woven fabric chassis layer(s). Suitable materials for an additional chassis layer include a spun-bonded non-woven web of synthetic fibers such as polypropylene, polyethylene or polyester fibers; a non-woven web of cellulosic fibers, textile fibers such as rayon fibers, cotton and the like, or a blend of cellulosic and textile fibers; a spun-bonded non-woven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers. The chassis layer may comprise a non-woven polyethylene or polypropylene sheet, a polyethylene film, or any other suitable garment material. The chassis layer may comprise a liquid pervious or liquid impervious material or may be zone-treated to be partially liquid pervious or impervious.

Absorbent core 16 may include one or a plurality of layers of material, such as an absorbent layer for storing fluids and an acquisition layer (in addition to acquisition members 13) for distributing fluids. Absorbent core 16 may be made from absorbent materials such as, for example, wood fibers, tow fibers, chemical wood pulp, or any other suitable liquid absorbing material, such as commercially available fluff pulp or fluffed bleached kraft softwood pulp. It is contemplated that absorbent core 16 may include a porous fibrous web and super absorbent particles (SAP). Absorbent core 16 may be a laminate material including outer tissue layers containing a high percentage by weight of SAP. Various suitable types of SAP may be employed with absorbent core 16 that are in accordance with the principles of the present disclosure, including for example modified polymers such as sodium neutralized cross-linked polyacrylates and polysaccharides including, for example, cellulose and starch and regenerated cellulose that are modified to be carboxylated, phosphonoalkylated, sulphoxylated or phosphorylated, causing the SAP to be highly hydrophilic. Such modified polymers may also be cross-linked to reduce their water-solubility. Commercially available SAPs include a starch modified superabsorbent polymer available under the trade name SANWET® from BASF Corporation, Portsmouth, Va., a superabsorbent derived from polypropenoic acid, available under the tradename DRYTECH® 520 SUPERABSORBENT POLYMER from The Dow Chemical Company, Midland Mich., AQUA KEEP manufactured by Seitetsu Kagaku Co., Ltd., ARASORB manufactured by Arakawa Chemical (U.S.A.) Inc., ARIDALL 1125 manufactured by Chemdall Corporation, FAVOR manufactured by Stockhausen Inc., HYSORB from BASF Atkienqesellshaft, Ludwigshafen, Germany, AQUA KEEP SA6OS, manufactured by Seitetsu Kagaku Co., Ltd., DIAWET, commercially available from Mitsubishi Chemicals, Japan, and FLOSORB, available from SNF Floerger, France, and AQUALIC, available from Nippon Shokubai, Osaka, Japan.

Absorbent core 16, and any additional layers associated therewith, may be folded, crimped, thermally bonded, or otherwise manipulated to provide additional benefits. It is contemplated that various types of folds may be employed to provide additional fluid handling capabilities, such as, for example, U shape, C shape, G shape, Z shape, or other shapes. Absorbent core 16 is elongated along longitudinal axis x, and extends in the direction of the side portions of training pants 10 along lateral axis y. Absorbent core 16 is rectangular, however, may alternatively have other configurations such as circular, elliptical, I shape or T shape. Absorbent core 16 may have channels, grooves or pockets, and may have varying thickness.

Top layer 12 includes top sheet panels that extend upwardly to form waste containment flaps 26 (FIG. 2). Waste containment flaps 26 may be treated with a suitable surfactant to modify their hydrophobicity/hydrophilicity as desired, and they may be treated with skin wellness ingredients to reduce skin irritation. Alternatively, waste containment flaps 26 may be formed as separate elements and then attached to top layer 12. Waste containment flaps 26 includes elastic elements 28 that facilitate extension of waste containment flaps 26 from top layer 12 to prevent leakage of fluid discharge and body waste. Training pants 10 also includes leg gathers 30, as well as waste containment flaps 26, for improving the fit with a wearer. Leg gathers 30 contract leg holes 32 around the wearer's legs (not shown) and body to prevent leakage. A barrier film 34 is disposed between core 16 and back/chassis layer 14.

The assemblies of top layer 12, absorbent core 16, acquisition members 18 and barrier film 34 are attached to back/chassis layer 14 by known processes, such as by ultrasonic bonding or by the use of lines of hot melt adhesive. The bond between the assembly and back/chassis layer 14 is reinforced by laterally-extending end strips 36 that are applied over the longitudinal ends of the assembly and bonded to the underlying structure of training pants 10. End strips 36 maintain the ends of leg gathers 32 in an inward or outward facing orientation. End strips 36 are fabricated from a fluid pervious non-woven material. It is contemplated that end strips 36 may be fluid impervious. End strips 36 prevent the longitudinal flow of fluid discharge and body waste from training pants 10.

Training pants 10 include waist elastics 38 and abdomen elastics 40 that contract the training pants 10 about the wearer's abdomen. Elastics 38, 40 may be applied in an inelastic state and then heat activated to cause them to become elasticized. Elastics 38, 40 and 28 may be made from natural or synthetic rubber, elastomers, LYCR.A® elastomer (available from E. I. DuPont de Nemours and Company, a business having offices in Wilmington, Del.), polyurethane, heat shrinkable polymer ribbons, or any other suitable elastic material or composite. Waist elastics 38 are located proximal to longitudinal ends 42 of back/chassis layer 14, thereby encircling a waist edge 44 of the fully assembled training pants 10.

Abdomen elastics 40 are disposed in training pants 10 between longitudinal ends 42 and leg opening cutouts 32 to position training pants 10 across the wearer's stomach. Abdomen elastics 40 are disposed between a pair of carrier layers 66, 68 to form elastic assemblies with abdomen elastics 40, which are attached to back/chassis layer 14. It is envisioned that abdomen elastics 40 may extend across the entire width of training pants 10. Elastics 38, 40, 28 may be joined to training pants 10 by the use of a flexible adhesive or other suitable joining method. Suitable adhesives include HL-1258 by H. B. Fuller Company of St. Paul, Miun., Findley 2031 and H2587-01 by Ato Findley Inc. of Wauwatosa, Wis., DISPOMELT® 34-5665 by National Starch Co. of Bridgewater, N.J., DISPOMELT® 34-578A by National Starch Co. of Bridgewater, N.J., and HL 1486UZP, which is available from H. B. Fuller Company of St. Paul, Minn.

Training pants 10 fastens front waist region 20 with rear waist region 24 to form a waist hole 70. A seal is formed whereby lateral edge portions 72 (FIG. 2) of back/chassis layer 14 are attached to one another to form side seals 74 (FIG. 1). Lateral edge portions 72 of back/chassis layer 14 are joined by various known methods to contain the various components and form training pants 10. Joining of lateral edge portions 72 defines leg holes 32 and longitudinal ends 42 of training pants 10 form waist edge 44. Training pants 10 are fully formed and can be manipulated to pull onto the wearer, similar to a pair of pants. Back/chassis layer 14 includes visual representation, such as, for example, applique 76 that provides a graphic representation on training pants 10. Appliquè 76 provides a visual representation for decoration, aesthetic purposes or to provide indicia for particular characteristics of training pants 10. Other forms of visual representation on training pants 10 are also contemplated such as color, printed patterns or embossing.

The invention also encompasses a method of making training pants 10 that includes providing a top layer material, a back layer material, a plurality of acquisition members and an absorbent core to an absorbent article forming station. The method also includes disposing the absorbent core between the top layer material and the back layer material at the absorbent article forming station. The method further includes disposing the plurality of acquisition members in parallel with a longitudinal length of the core.

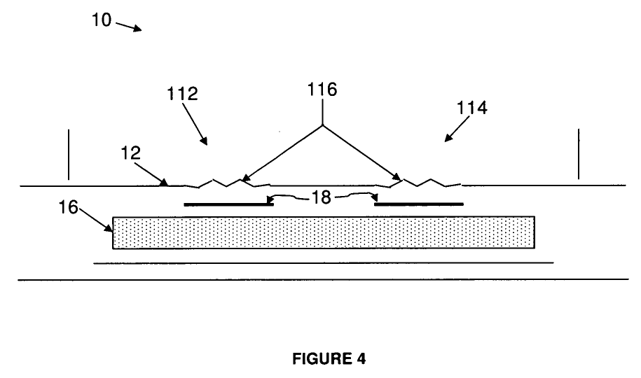

In an alternate embodiment of training pants 10, as shown in FIG. 4, a portion 112 of top layer 12 is mechanically deformed to form a predetermined pattern 114. Predetermined pattern 114 includes puckers 116 that are in spatial alignment with acquisition members 18. Acquisition members 18 are disposed in parallel with a longitudinal length of core 16 and in spatial alignment with puckers 116 in a configuration that retards strikethrough time. This allows the wearer to feel wetter for a shorter period of time, while allowing dry out to maintain health of the skin of the wearer. This configuration advantageously promotes toilet training, and contains fluid discharge and body waste. It is contemplated that predetermined pattern 114 may alternatively include folds and/or pleats.

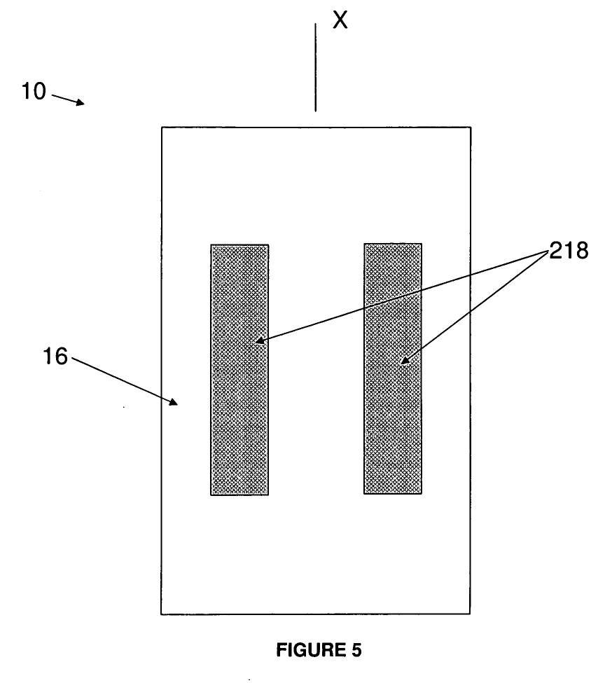

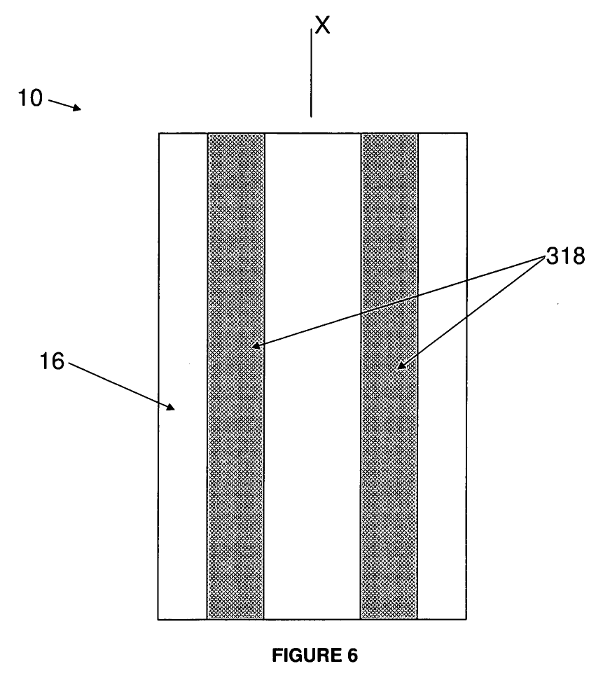

In another alternate embodiment of training pants 10, as shown in FIG. 5, acquisition members 218 are longitudinally centered on absorbent core 16. Acquisition members 218 extend partially along the length of core 16, along longitudinal axis x. It is envisioned that acquisition members 218 may be disposed at various positions along core 16. In another alternate embodiment of training pants 10, as shown in FIG. 6, acquisition members 318 extend along the entire length of absorbent core 16, along longitudinal axis x.

The claims are intended to cover all of the foregoing classes of absorbent articles, without limitation, whether disposable, unitary or otherwise. These classifications are used interchangeably throughout the specification, but are not intended to limit the claimed invention. The invention will be understood to encompass, without limitation, all classes of absorbent articles, including those described above.

The invention of the present disclosure may be embodied in other specific forms without departing from the spirit or essential characteristics thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed is:

1. An absorbent article comprising:
a liquid pervious top layer;
a liquid impervious back layer;
an absorbent core disposed between the top layer and the back layer; and
a plurality of acquisition members being disposed between the top layer and the core, the acquisition members being disposed in a configuration that retards strikethrough time.

2. An absorbent article as recited in claim 1, further comprising four acquisition members.

3. An absorbent article as recited in claim 1, wherein the acquisition members extend an entire length of the core.

4. An absorbent article as recited in claim 1, wherein the acquisition members extend only a portion of the length of the core.

5. An absorbent article as recited in claim 1, wherein the acquisition members include non-woven material.

6. An absorbent article as recited in claim 1, wherein the acquisition members provide visual indicia.

7. An absorbent article as recited in claim 1, wherein the acquisition members are in contacting engagement.

8. An absorbent article as recited in claim 1, wherein the acquisition members are in spaced apart relation.

9. An absorbent article as recited in claim 1, wherein the acquisition members are spaced apart a distance in a range of 1 mm to 30 mm.

10. An absorbent article as recited in claim 1, wherein each of the acquisition members are a width in a range of 10 mm to 70 mm wide.

11. An absorbent article as recited in claim 1, wherein a portion of the top layer is deformed to form a predetermined pattern.

12. An absorbent article as recited in claim 11, wherein the acquisition members are designed parallel with a longitudinal length of the core.

13. An absorbent article as recited in claim 12, wherein the predetermined pattern is in spatial alignment with the acquisition members.

14. An absorbent article as recited in claim 1, further comprising a film layer disposed between the core and the back layer.

15. A disposable absorbent article comprising:
a liquid pervious top layer, a portion of the top layer being deformed to form a predetermined pattern;
a liquid impervious back layer;
an absorbent core disposed between the top layer and the back layer; and
a plurality of acquisition members being disposed between the top layer and the core, the acquisition members being disposed parallel with a longitudinal length of the core in a configuration that retards strikethrough time.

16. An absorbent article as recited in claim 15, wherein the acquisition members extend an entire length of the core.

17. An absorbent article as recited in claim 15, wherein the acquisition members provide a visual indicia.

18. An absorbent article as recited in claim 15, wherein the acquisition members are in contacting engagement.

19. An absorbent article as recited in claim 15, wherein the acquisition members are in spaced apart relation.

20. An absorbent article as recited in claim 15, further comprising a film layer disposed between the core and the back layer.

21. An absorbent article as recited in claim 15, wherein the acquisition members are in spatial alignment with the predetermined pattern.

22. A disposable training absorbent article comprising:
a liquid pervious top layer having leg gathers disposed adjacent lateral edges thereof, a portion of the top layer being mechanically deformed to form a predetermined pattern of puckers;
a liquid impervious back layer;
an absorbent core disposed between the top layer and the back layer;
a plurality of acquisition members being spaced apart and disposed between the top layer and the core, the acquisition members being disposed parallel with a longitudinal length of the core and in spatial alignment with the puckers in a configuration that retards strikethrough time; and
a film layer being disposed between the core and the back layer.
